SEQUENCE LISTING (26 sequences)

SEQ ID NO: 1 - DNA, 3720 bp, Cucumis melo
tcagccgttggttgtgagatgcaagtgatacctgatccaaccactgttcatttccatctaccaactaacctttcagtacggattcacagagaatacagtgagtttattgcagaacttgtcagcaattttccccatgaaaaagatggaatcctcaaattttatggggattgttggaaggttagtttccattgagatgactgcatcttcctccagatttctatctccacgaccattgactcacatgatgagcttgattgcatgtgtagatattccaatgctttaaactcattggaactaaaatcactggaggagccaatatatctttttggacagttctttcagaagcctcttgcatgcctgacacttggtaatgatgtctttatttatttattaatttttaattattgcaatcacagtaagatttttgtaggttgaaataaccaataaagaatataatggaatctttaattagatttttgctgcattcatactactcctcaacgccccccacccacatgatattttccatctaagaagaaatgctctgctgcttatttgctaagcagtcaattttctttgaatgcgttggctgcattctactgcacggcatagaaatcacaattacttttcattcttatcacctatgctcattcattaacttacatgtaaaatatttaaatatatttgcattgcagcatattacttgcctcaaaatgctggagacttggctcggaagtacattaaggatccccgtctgttgtcctttattgatgcagaggtgagcctagagaattgattaagaatatttgttagtttgttattgtttatttagaaattatagggttttcttttcctactccatacatttatccagacactaatctatatgtggtggttttcagtgttttatcgttagcacagtgaatgctttgcaaacaccaatgataaatgcggccatggtaagacttaatgagttgtggtcaaccttttacttcagttctttaatctgtctcgattcactgaatgctgtctgcttcaggttttatgtgacaggcattttggtgggataaattaccctattggtggtgttggtggaattgcaaagtccttggcaaggggactggttgatcatggaagctcaataatgtacaaagcaaatgtgacaaagataataactgaaaatggcaaagctgtgagtatttattttcagcatattacaattcaaaaaagggattgagatttcctttgacttcagaaaatattattgttgtcttaactagttgggttatgctcgacttagttatatgtttagatttgtgtttattgtgtccagttttattatttacttggtttattttgtaggtaggcgtgaagctgtctgatggaagggagttctttgctaaaactattgtatcgaatgctactagatgggatacctttggttagttcttctcaagttctgtagcagaatttttactatattgcttggctgattgtttcatctctctctcatgtccttaccagattctagtaattactgaatatgtaagagctttgttttatttaaggatagctgttgaaaggagtggaccttcccaaggaagaggagaactttcagaaactttatgttaaggccccatctttcctttcaattcatatgggagtgaaagctgaggttttaccgccggatacagattgtcaccattttgtgcttgaggtagtggatgctcttgctagttgtaatcttcaacaatttcagttaatttctcatggaattgctgtggaaccgattgtttccttctttcttgttgtattattagagtgattggagaaggttagaggagccatatggaagcatctttctgagcatcccgactgttcttgatccatcattagctccagatggacgtcatattcttcacatttttactacttcttccatggaggattgggaggtgagtttaattttcttaataagttcatatttaattaattttaaaaacaaacatttggtaatgaagtttcagttgtcgcactctaataatatttgtgcataacggatgcccactaatatgcggagtcccctccacaaagccagtttggtaaacaataagaattttaagctattaaaattttcaacctgactacccgagagcttgaatctagaacatggagaaaactcttgtaaatgacaatagacactcaaatcatggcaaatcttaccaccagggcattccttgatttttagtaatagtctttaatttcgtctcccatcttcctgtttttcacactccacatgattagttgtttcttcctacaccaggggctctccagggaagagtacgaagcaaagaaggcgctgatagcagatggaattatcactagactcgagaagaagctatttccagggttaaaatcatctattgattttatggaggtataagttaggttctaattttctactaaacaatcttgctgatttttaaaagcatcaatgaatggggaatgtttgccacctaaagctgaaccataccttacagaatagcatggtgcatttgtaaaaatccgaattaattgttgaagaagtttgtcgcaacatgaatgataataattaaaacagtgagttatattatccagtagccatgtgaactttgtgggagtatactattccttctgattatgtttcatgaaaggacctacacattcatctttccatatgcagggcaccaaatctcatcggtctaattgtttattttatttttctcgatcaatgaggctgtattattttatcttttaataatgcccaccttggtacttgcacgtgatgttgaaaattgtggaattggaacgtacacttcaagttgaatatcgtgaaaatattacttttttctggttagaagctttaatcatcccgttaaagttgattgtgacttcttgtaacattcataagcacagtactagattgcattaacttgaggccatagagtaactctgaatgatatgttggttgaaaattttctgaaacgatgacaggttgggacgccaaagacacacaggcgattcctagcccgtgataatggtacctatggaccaatgccacgaggcactcctaagggattgcttggaatgccatttaatacaactgtgagttgatcgaatgctaacctctcgtctatgttctttttttacatccaactgaagggccaccaatgattacctgcagggtatagatgggctgtattgtgttggtgatagttgctttcctggccaaggagtaattgctgtagccttttccggagtgatgtgtgcacaccgagtagctgcagatattggtgagctatagaccatgatttaattttccagattcttggtgattatttaccaacacaggtagtctatcgaaacaattttagctcaatcaatacaaagagattaaataacaattgcagttaggtatgacatactttaagatctgaacttcttcgttggttgtcatcccggaaatgaattgcttgtttaatggaccacacaacacattgcttcaagttctaacttgtcaacgtcatgtagtttcttcttagttaggctagactttcacaacctcctgattctttctactttgacagggcttgaaaagaagtcccccattttggatgcggcccttcttcggctacttggttggttgaggaccttggcctgaattttgatgtgagaatgcac

SEQ ID NO: 2 - DNA, 1380 bp, Cucumis melo
aatttaattaacacaagctttgtcagccgttggttgtgagatgcaagtgatacctgatccaaccactgttcatttccatctaccaactaacctttcagtacggattcacagagaatacagtgagtttattgcagaacttgtcagcaattttccccatgaaaaagatggaatcctcaaattttatggggattgttggaagatattcaatgctttaaactcattggaactaaaatcactggaggagccaatatatctttttggacagttctttcagaagcctcttgcatgcctgacacttgcatattacttgcctcaaaatgctggagacttggctcggaagtacattaaggatccccgtctgttgtcctttattgatgcagagtgttttatcgttagcacagtgaatgctttgcaaacaccaatgataaatgcggccatggttttatgtgacaggcattttggtgggataaattaccctattggtggtgttggtggaattgcaaagtccttggcaaggggactggttgatcatggaagctcaataatgtacaaagcaaatgtgacaaagataataactgaaaatggcaaagctgtaggcgtgaagctgtctgatggaagggagttctttgctaaaactattgtatcgaatgctactagatgggatacctttggatagctgttgaaaggagtggaccttcccaaggaagaggagaactttcagaaactttatgttaaggccccatctttcctttcaattcatatgggagtgaaagctgaggttttaccgccggatacagattgtcaccattttgtgcttgagagtgattggagaaggttagaggagccatatggaagcatctttctgagcatcccgactgttcttgatccatcattagctccagatggacgtcatattcttcacatttttactacttcttccatggaggattgggaggggctctccagggaagagtacgaagcaaagaaggcgctgatagcagatggaattatcactagactcgagaagaagctatttccagggttaaaatcatctattgattttatggaggttgggacgccaaagacacacaggcgattcctagcccgtgataatggtacctatggaccaatgccacgaggcactcctaagggattgcttggaatgccatttaatacaactggtatagatgggctgtattgtgttggtgatagttgctttcctggccaaggagtaattgctgtagccttttccggagtgatgtgtgcacaccgagtagctgcagatattgggcttgaaaagaagtcccccattttggatgcggcccttcttcggctacttggttggttgaggaccttggcctgaattttgatgtgagaatg

SEQ ID NO: 3 - protein, 440 aa, Artificial sequence
  Deduced protein sequence of the wild type CRTISO
MQVIPDPTTVHFHLPTNLSVRIHREYSEFIAELVSNFPHEKDGILKFYGDCWKIFNALNSLELKSLEEPIYLFGQFFQKPLACLTLAYYLPQNAGDLARKYIKDPRLLSFIDAECFIVSTVNALQTPMINAAMVLCDRHFGGINYPIGGVGGIAKSLARGLVDHGSSIMYKANVTKIITENGKAVGVKLSDGREFFAKTIVSNATRWDTFGKLLKGVDLPKEEENFQKLYVKAPSFLSIHMGVKAEVLPPDTDCHHFVLESDWRRLEEPYGSIFLSIPTVLDPSLAPDGRHILHIFTTSSMEDWEGLSREEYEAKKALIADGIITRLEKKLFPGLKSSIDFMEVGTPKTHRRFLARDNGTYGPMPRGTPKGLLGMPFNTTGIDGLYCVGDSCFPGQGVIAVAFSGVMCAHRVAADIGLEKKSPILDAALLRLLGWLRTLA

SEQ ID NO: 4 - protein, 211 aa, Artificial sequence
  Deduced protein sequence of CEM 3285 Mutated CRTISO (ORF 1)
MQVIPDPTTVHFHLPTNLSVRIHREYSEFIAELVSNFPHEKDGILKFYGDCWKIFNALNSLELKSLEEPIYLFGQFFQKPLACLTLAYYLPQNAGDLARKYIKDPRLLSFIDAECFIVSTVNALQTPMINAAMVLCDRHFGGINYPIGGVGGIAKSLARGLVDHGSSIMYKANVTKIITENGKAVGVKLSDGREFFAKTIVSNATRWDTFG

SEQ ID NO: 5 - protein, 229 aa, Artificial sequence
  Deduced protein sequence of CEM 3285 Mutated CRTISO 5bp deletion (ORF 1)
MQVIPDPTTVHFHLPTNLSVRIHREYSEFIAELVSNFPHEKDGILKFYGDCWKIFNALNSLELKSLEEPIYLFGQFFQKPLACLTLAYYLPQNAGDLARKYIKDPRLLSFIDAECFIVSTVNALQTPMINAAMVLCDRHFGGINYPIGGVGGIAKSLARGLVDHGSSIMYKANVTKIITENGKAVGVKLSDGREFFAKTIVSNATRWDTFAVERSGPSQGRGELSETLC

SEQ ID NO: 6 - RNA, 9 nt, Artificial sequence
  An exemplary shRNA loop sequence
uucaagaga

SEQ ID NO: 7 - RNA, 9 nt, Artificial sequence
  An exemplary shRNA loop sequence
uuuguguag

SEQ ID NO: 8 - DNA, 23 bp, Artificial sequence
  Exemplary CRISTO targeting siRNA sequence
gcctgacacttgcatattacttg

SEQ ID NO: 9 - DNA, 22 bp, Artificial sequence
  A single strand DNA oligonuceotide
gatggagctctacgccgatgtc

SEQ ID NO: 10 - DNA, 22 bp, Artificial sequence
  A single strand DNA oligonuceotide
cctccctggcacatgaaattag

SEQ ID NO: 11 - DNA, 20 bp, Artificial sequence
  A single strand DNA oligonuceotide
aggggactggttgatcatgg

SEQ ID NO: 12 - DNA, 22 bp, Artificial sequence
  A single strand DNA oligonuceotide
gcacaaaatggtgacaatctgt

SEQ ID NO: 13 - DNA, 1323 bp, Artificial sequence
  CRISTO antisense polynucleotide
tcaggccaaggtcctcaaccaaccaagtagccgaagaagggccgcatccaaaatgggggacttcttttcaagcccaatatctgcagctactcggtgtgcacacatcactccggaaaaggctacagcaattactccttggccaggaaagcaactatcaccaacacaatacagcccatctataccagttgtattaaatggcattccaagcaatcccttaggagtgcctcgtggcattggtccataggtaccattatcacgggctaggaatcgcctgtgtgtctttggcgtcccaacctccataaaatcaatagatgattttaaccctggaaatagcttcttctcgagtctagtgataattccatctgctatcagcgccttctttgcttcgtactcttccctggagagcccctcccaatcctccatggaagaagtagtaaaaatgtgaagaatatgacgtccatctggagctaatgatggatcaagaacagtcgggatgctcagaaagatgcttccatatggctcctctaaccttctccaatcactctcaagcacaaaatggtgacaatctgtatccggcggtaaaacctcagctttcactcccatatgaattgaaaggaaagatggggccttaacataaagtttctgaaagttctcctcttccttgggaaggtccactcctttcaacagctttccaaaggtatcccatctagtagcattcgatacaatagttttagcaaagaactcccttccatcagacagcttcacgcctacagctttgccattttcagttattatctttgtcacatttgctttgtacattattgagcttccatgatcaaccagtccccttgccaaggactttgcaattccaccaacaccaccaatagggtaatttatcccaccaaaatgcctgtcacataaaaccatggccgcatttatcattggtgtttgcaaagcattcactgtgctaacgataaaacactctgcatcaataaaggacaacagacggggatccttaatgtacttccgagccaagtctccagcattttgaggcaagtaatatgcaagtgtcaggcatgcaagaggcttctgaaagaactgtccaaaaagatatattggctcctccagtgattttagttccaatgagtttaaagcattgaatatcttccaacaatccccataaaatttgaggattccatctttttcatggggaaaattgctgacaagttctgcaataaactcactgtattctctgtgaatccgtactgaaaggttagttggtagatggaaatgaacagtggttggatcaggtatcacttgcat

SEQ ID NO: 14 - protein, 228 aa, Artificial sequence
  Deduced protein sequence of CEM 3285 Mutated CRTISO (ORF 2)
LLKGVDLPKEEENFQKLYVKAPSFLSIHMGVKAEVLPPDTDCHHFVLESDWRRLEEPYGSIFLSIPTVLDPSLAPDGRHILHIFTTSSMEDWEGLSREEYEAKKALIADGIITRLEKKLFPGLKSSIDFMEVGTPKTHRRFLARDNGTYGPMPRGTPKGLLGMPFNTTGIDGLYCVGDSCFPGQGVIAVAFSGVMCAHRVAADIGLEKKSPILDAALLRLLGWLRTLA

SEQ ID NO: 15 - protein, 13 aa, Artificial sequence
  Deduced protein sequence of CEM 3285 Mutated CRTISO 5bp deletion (ORF 2)
GPIFPFNSYGSES

SEQ ID NO: 16 - protein, 13 aa, Artificial sequence
  Deduced protein sequence of CEM 3285 Mutated CRTISO 5bp deletion (ORF 3)
GFTAGYRLSPFCA

SEQ ID NO: 17 - protein, 1 aa, Artificial sequence
  Deduced protein sequence of CEM 3285 Mutated CRTISO 5bp deletion (ORF 4)
E

SEQ ID NO: 18 - protein, 19 aa, Artificial sequence
  Deduced protein sequence of CEM 3285 Mutated CRTISO 5bp deletion (ORF 5)
LEKVRGAIWKHLSEHPDCS

SEQ ID NO: 19 - protein, 43 aa, Artificial sequence
  Deduced protein sequence of CEM 3285 Mutated CRTISO 5bp deletion (ORF 6)
SIISSRWTSYSSHFYYFFHGGLGGALQGRVRSKEGADSRWNYH

SEQ ID NO: 20 - protein, 13 aa, Artificial sequence
  Deduced protein sequence of CEM 3285 Mutated CRTISO 5bp deletion (ORF 7)
TREEAISRVKIIY

SEQ ID NO: 21 - protein, 16 aa, Artificial sequence
  Deduced protein sequence of CEM 3285 Mutated CRTISO 5bp deletion (ORF 8)
FYGGWDAKDTQAIPSP

SEQ ID NO: 22 - protein, 11 aa, Artificial sequence
  Deduced protein sequence of CEM 3285 Mutated CRTISO 5bp deletion (ORF 9)
WYLWTNATRHS

SEQ ID NO: 23 - protein, 7 aa, Artificial sequence
  Deduced protein sequence of CEM 3285 Mutated CRTISO 5bp deletion (ORF 10)
GIAWNAI

SEQ ID NO: 24 - protein, 11 aa, Artificial sequence
  Deduced protein sequence of CEM 3285 Mutated CRTISO 5bp deletion (ORF 11)
YNWYRWAVLCW

SEQ ID NO: 25 - protein, 27 aa, Artificial sequence
  Deduced protein sequence of CEM 3285 Mutated CRTISO 5bp deletion (ORF 12)
LLSWPRSNCCSLFRSDVCTPSSCRYWA

SEQ ID NO: 26 - protein, 22 aa, Artificial sequence
  Deduced protein sequence of CEM 3285 Mutated CRTISO 5bp deletion (ORF 13)
KEVPHFGCGPSSATWLVEDLGL

(19) United States
(12) Patent Application Publication
(10) Pub. No.: US 2012/0324597 A1
(43) Pub. Date: Dec. 20, 2012

(54) MELON PLANTS COMPRISING TETRA-CIS-LYCOPENE

(75) Inventors: Yaakov Tadmor, Timrat (IL); Yosef Burger, Haifa (IL); Nurit Katzir, Kiryat-Tivon (IL); Efraim Lewinsohn, Timrat (IL); Vitaly Portnoy, Nesher (IL); Tamar Lavee, Moshav Sde Yaakov (IL); Ayala Meir, Givat Elah (IL); Uzi Saar, Kiryat-Tivon (IL); Arthur A. Schaffer, Hashmonaim (IL)

(73) Assignee: THE STATEOF ISRAEL,MINISTRY OF AGRICULTURE & RURAL DEVELOP., AGRICULTURAL RESEARCH ORGANIZATION, Beit-Dagan (IL)

(21) Appl. No.: 13/580,164

(22) PCT Filed: Feb. 22, 2011
(86) PCT No.: PCT/IL11/00177

§ 371 Date: Aug. 21, 2012

Related U.S. Application Data
(60) Provisional application No. 61/306,589, filed on Feb. 22, 2010.

(51) Int. Cl. A01H 5/00; A01H 1/06; A01D 45/00; A01G 1/00; A01C 11/00; A01H 1/02; A01H 5/10

(52) U.S. Cl. 800/260; 800/309; 800/276; 47/58.1 FV

(57) ABSTRACT

A Cucumis melo plant is disclosed, wherein a flesh of a fruit of the plant comprises tetra-cis-lycopene (pro-lycopene). Methods of generating same are also disclosed.

### FIELD AND BACKGROUND OF THE INVENTION

The present invention, in some embodiments thereof, relates to melon plants comprising tetra-cis lycopene as the major fruit colorant and methods of generating same.

Carotenoid pigments are essential components in all photosynthetic organisms. They assist in harvesting light energy and protect the photosynthetic apparatus against harmful reactive oxygen species that are produced by overexcitation of chlorophyll. They also furnish distinctive yellow, orange, and red colors to fruit and flowers to attract animals. Additionally, carotenoids are phytonutrients with a widely claimed range of health-benefiting activities, including prevention of major disease such as cancer, coronary disease and age related eye malfunction.

Carotenoids are mainly 40-carbon isoprenoids, which consist of eight isoprene units. The polyene chain in carotenoids contains up to 15 conjugated double bonds, a feature that is responsible for their characteristic absorption spectra and specific photochemical properties. These double bonds enable the formation of cis-trans geometric isomers in various positions along the molecule. Indeed, although the bulk of carotenoids in higher plants occur in the all-trans configuration, different cis-isomers exist as well, but in small proportions.

In plants, carotenoids are synthesized within the plastids from the central isoprenoid pathway (Hirschberg, 2001, Curr Opin Plant Biol 4, 210-218; FIG. 1). The first carotenoid in the pathway is the colorless phytoene, which is produced by the enzyme phytoene synthase (PSY) through a condensation of two molecules of geranylgeranyl diphosphate. Four double bonds are introduced subsequently into phytoene by two enzymes, phytoene desaturase (PDS) and ξ-carotene desaturase (ZDS), each catalyzing two symmetric dehydrogenation steps to yield ξ-carotene and all-trans-lycopene, the red pigment of tomato and watermelons.

Lycopene is the substrate for specific cyclases, while β-cyclization of both ends of lycopene yields β-carotene, an orange pigment. All major plant carotenoids appear in their trans form through the activity of carotenoid isomerase (CRTISO). If CRTISO is non-functional, the orange pigment pro-lycopene (tetra-cis-lycopene) is accumulated since the cyclases are specific to all-trans lycopene (FIG. 1).

Melons, Cucumis melo, belong to the Cucurbitaceae family. In Western society, the melon fruit is consumed when the fruit is fully matured, as a desert. When matured, the flesh of the fruit exhibits a wide range of colors, including white, cream, green, yellow, orange and combinations thereof. Melon fruit pigments are carotenoids and chlorophylls. In a comprehensive screening of carotenoids in more than 200 accessions, representing the widely known melon germplasm, it was found that orange melons accumulate β-carotene as their major pigment while green melons mostly accumulate chlorophylls and a combination of two chloroplastic carotenoids, β-carotene and lutein (Burger et al., 2006, Israel J Plant Sci, 54:233-242).

Fruits that accumulate pro-lycopene as their major carotenoid include tomato and watermelon (Tadmor et al [Food Research International 38 (2005) 837-841]. Linkage of a dysfunctional carotenoid isomerase (CRTISO) to fruit pro-lycopene accumulation was first reported by Isaacson et al. [The Plant Cell, 14 (2002) 333-342].

### SUMMARY OF THE INVENTION

According to an aspect of some embodiments of the present invention there is provided a Cucumis melo plant, wherein a flesh of a fruit of the plant comprises tetra-cis-lycopene (pro-lycopene).

According to an aspect of some embodiments of the present invention there is provided a Cucumis melo plant, having a genome, the genome comprising at least one allele of CRTISO having a loss of function mutation.

According to an aspect of some embodiments of the present invention there is provided a seed derived from the plant of the present invention.

According to an aspect of some embodiments of the present invention there is provided a fruit derived from the plant of the present invention.

According to an aspect of some embodiments of the present invention there is provided a pollen derived from the plant of the present invention.

According to an aspect of some embodiments of the present invention there is provided an ovule derived from the plant of the present invention.

According to an aspect of some embodiments of the present invention there is provided a cell derived from the plant of the present invention.

According to an aspect of some embodiments of the present invention there is provided a cell culture comprising the cell of the present invention.

According to an aspect of some embodiments of the present invention there is provided a method for producing a hybrid melon seed comprising crossing a first parent melon plant with a second parent melon plant and harvesting the resultant hybrid F₁ seed, wherein at least one of the first or the second parent melon plant is the plant of the present invention.

According to an aspect of some embodiments of the present invention there is provided a hybrid melon seed produced by the method comprising crossing a first parent melon plant with a second parent melon plant and harvesting the resultant hybrid F₁ seed, wherein at least one of the first or the second parent melon plant is the plant of the present invention.

According to an aspect of some embodiments of the present invention there is provided a hybrid melon plant, or parts thereof, produced by growing the hybrid melon seed produced by the method comprising crossing a first parent melon plant with a second parent melon plant and harvesting the resultant hybrid F₁ seed, wherein at least one of the first or the second parent melon plant is the plant of the present invention.

According to an aspect of some embodiments of the present invention there is provided a seed produced by growing the hybrid melon plant produced by growing the hybrid melon seed produced by the method comprising crossing a first parent melon plant with a second parent melon plant and harvesting the resultant hybrid F₁ seed, wherein at least one of the first or the second parent melon plant is the plant of the present invention.

According to an aspect of some embodiments of the present invention there is provided a method of generating Cucumis melo plant, wherein a flesh of a fruit of the plant comprises tetra-cis-lycopene (pro-lycopene), the method comprising down-regulating an amount and/or activity of carotenoid isomerase (CRTISO) in a Cucumis melo plant, thereby generating the plant.

According to an aspect of some embodiments of the present invention there is provided a method of generating a Cucumis melo fruit having a flesh which comprises a greater amount of tetra-cis-lycopene (pro-lycopene) than β-carotene and/or having at least one allele of CRTISO having a loss of function mutation, the method comprising:

(a) seeding seeds of the Cucumis melo fruit, and/or planting seedlings of the seeds;

(b) growing plants generated from the seeds or the seedlings; and

(c) harvesting the Cucumis melo fruit of the plants, thereby generating the Cucumis melo fruit.

According to an aspect of some embodiments of the present invention there is provided a seed of a Cucumis melo line CEM 3285, a sample of the seed of which has been deposited under NCIMB Accession Number 41710 on 16 April, 2010.

According to an aspect of some embodiments of the present invention there is provided a plant of Cucumis melo line CEM 3285, a sample of the seed of which has been deposited under NCIMB Accession Number 41710 on 16 Apr., 2010.

According to some embodiments of the invention, the flesh of the fruit of the plant comprises a greater amount of tetra-cis-lycopene (pro-lycopene) than β-carotene.

According to some embodiments of the invention, the plant has a genome, the genome comprises at least one allele of CRTISO having a loss of function mutation.

According to some embodiments of the invention, the plant comprises a nucleic acid construct, the nucleic acid construct comprising a nucleic acid sequence encoding a polynucleotide agent which down-regulates an expression of CRTISO and a cis-acting regulatory element capable of directing an expression of the polynucleotide agent in the plant.

According to some embodiments of the invention, the polynucleotide agent is an siRNA or a ribozyme.

According to some embodiments of the invention, a flesh of a fruit of the plant comprises pro-lycopene.

According to some embodiments of the invention, the flesh of the fruit of the plant comprises a greater amount of tetra-cis-lycopene (pro-lycopene) than β-carotene.

According to some embodiments of the invention, the plant is devoid of carotenoid isomerase catalytic activity.

According to some embodiments of the invention, each allele of the CRTISO carries at least one loss of function mutation.

According to some embodiments of the invention, the CRTISO is in a homozygous form.

According to some embodiments of the invention, the CRTISO is in a heterozygous form.

According to some embodiments of the invention, the plant is a stable parental line.

According to some embodiments of the invention, the plant is a hybrid generated by crossing two parental lines.

According to some embodiments of the invention, the plant further comprises an additional trait consisting of herbicide resistance, insect resistance, resistance to bacterial, fungal or viral disease, male sterility and improved nutritional value.

According to some embodiments of the invention, the plant further comprises an additional trait selected from at least one type of disease resistance and at least one type of stress resistance.

According to some embodiments of the invention, the down-regulating is effected by chemical mutagenesis.

According to some embodiments of the invention, the down-regulating is effected by introducing into a Cucumis melo plant a nucleic acid construct, the nucleic acid construct comprising a nucleic acid sequence encoding a polynucleotide agent which down-regulates an expression of the CRTISO and a cis-acting regulatory element capable of directing an expression of the polynucleotide agent in the plant.

According to some embodiments of the invention, the polynucleotide agent is an siRNA or a ribozyme.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic presentation of β-carotene biosynthesis.

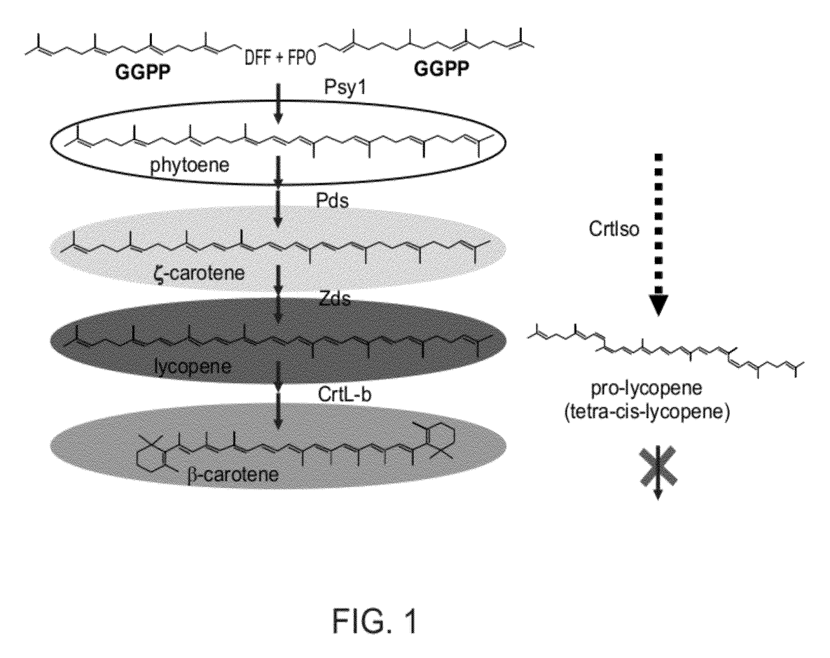

FIG. 2 is a photograph of orange melon fruits following chemical mutagenesis (CEM 3285). The upper half fruit accumulates β-carotene while the lower two halves are accumulating pro-lycopene as their major fruit carotenoid.

FIG. 3 is an HPLC chromatogram of mutated (top) and wild-type (bottom) CEM 3285 M₂ fruits.

FIG. 4 is a photograph of Mutated (left) and wild-type (right) plantlets of CEM 3285.

FIG. 5 is a photograph of plantlets of CEM 3285-13, a line stabilized for the induced mutation.

FIG. 6 is a photograph showing the cross section of ovary of mutated female flower (left) and wild-type female flower (right)

FIG. 7 is a photograph showing male flowers of wildtype (up) and mutated (down) flowers showing the petal's color differences

FIG. 8 is a genomic DNA sequence of the mutated carotenoid isomerase (CRTISO) gene. The first ATG is highlighted in green, Introns are colored yellow, the A to T transversion is marked in red, the five base deletion of the mis-spliced mRNA are underlined and the original STOP codon is highlighted in red (SEQ ID NO: 1).

FIG. 9 is the cDNA sequence of the mutated CRTISO gene. The first ATG is highlighted in green, the transversed T from A is marked in red, the resulting immature STOP codon is highlighted in yellow, the 5 bases deleted when mis-splicing occurs are underlined and the original STOP codon is highlighted in red (SEQ ID NO: 2).

FIGS. 10A-C are the deduced amino acids translated from the different mRNA. FIG. 10A: Native CRTISO. FIG. 10B: mutated protein of CRTISO when full length mRNA is transcribed. Transversion of A to T causes the appearance of immature STOP codon, highlighted in red, and thus the yellow highlighted protein sequence is not translated. FIG. 10C: The deleted mis-spliced mRNA is translated with a frame shift, highlighted in light blue, and immature STOP codon, highlighted in red. The yellow highlighted protein sequence is not translated.

FIG. 11 is a graphical representation of qRT-PCR analysis of CRTISO gene in developing fruits and in leaves of wild type plant (dark green and orange) or CEM 3285 (light green and yellow). The numbers designate days after pollination (DAP).

### DESCRIPTION OF SPECIFIC EMBODIMENTS OF THE INVENTION

The present invention, in some embodiments thereof, relates to melon plants comprising fruit tetra-cis lycopene and methods of generating same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Among Cucurbitaceae, Cucumis melo is one of the most important cultivated cucurbits. They are grown primarily for their fruit, which generally have a great diversity in size (50 g to 15 kg), flesh color (orange, green, white, and pink), rind color (green, yellow, white, orange, red, and gray), shape (round, flat, and elongated), and dimension (4 to 200 cm).

Lycopene is a naturally occurring carotenoid rarely found in fruits and vegetables. It is associated with antioxidant status, gap junction formation, and inhibition of cholesterol synthesis. Epidemiological studies suggest that high lycopene intakes are associated with decreased risks for cancer and heart disease, with an especially strong correlation with prostate cancer. In vitro studies show that lycopene inhibits growth of human endometrial, lung, and mammary cancer cells much more effectively than β-carotene Animal studies show that lycopene can inhibit brain and breast tumorigenesis.

Several research groups have suggested that cis-isomers of lycopene are better absorbed than the all-trans form because of the shorter length of the cis-isomer, the greater solubility of cis-isomers in mixed micelles, and/or as a result of the lower tendency of cis-isomers to aggregate [Burri et al., International Journal of Food Sciences and Nutrition (2008) 1-16].

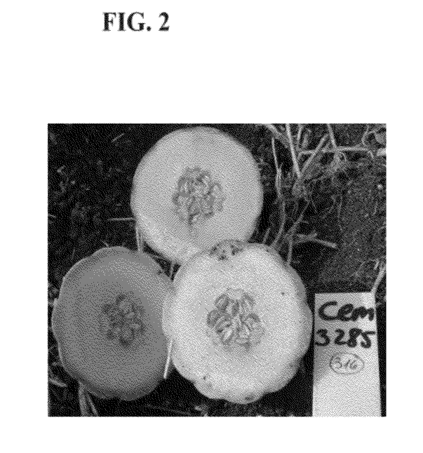

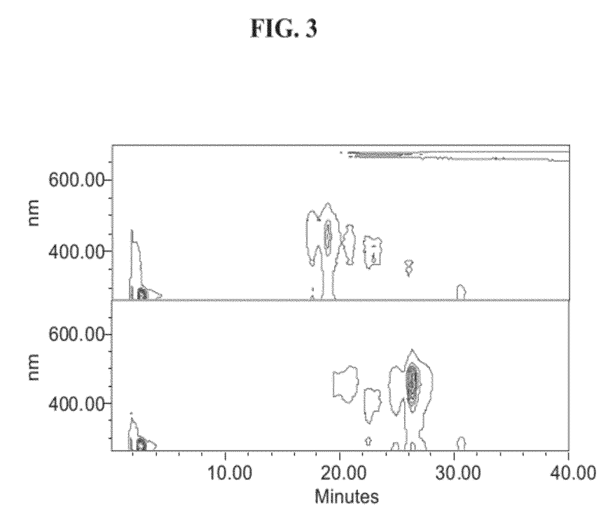

Whilst attempting to create novel variations of melon plant, the present inventions treated melon seeds with the chemical mutagen ethyl methanesulfonate (EMS) and selected melon with an unusual orange color (FIG. 2). HPLC analysis of carotenoids in the mutated fruit revealed altered carotenoid pattern compared to wild-type. The major carotenoid in the mutated fruit was tetra-cis-lycopene (pro-lycopene) while the wild-type fruits accumulated β-carotene as the major pigment (FIG. 3).

Since studies suggest that some cis-lycopene isomers are more bioavailable than the trans-lycopene isomer, the Cucumis melo of the present invention may be healthier or contribute different health benefits than naturally occurring Cucumis melo.

Analysis of the genomic DNA extracted from the mutated plants revealed that the gene encoding carotenoid isomerase (CRTISO) was mutated.

Thus, according to one aspect of the present invention there is provided a Cucumis melo plant, wherein a flesh of a fruit of the plant comprises tetra-cis-lycopene (pro-lycopene).

The term “plant” as used herein encompasses whole plants, ancestors and progeny of the plants and plant parts, including seeds, fruits, shoots, stems, roots (including tubers), and plant cells, tissues and organs. The plant may be in any form including suspension cultures, embryos, meristematic regions, callus tissue, leaves, gametophytes, sporophytes, pollen, ovules and microspores.

Typically, the Cucumis melo plant comprises a greater amount of tetra-cis-lycopene (pro-lycopene) than β-carotene.

According to still another embodiment of this aspect of the present invention the fruit flesh comprises at least 2 times, at least 4 times, at least 10 times the amount of tetra-cis-lycopene (pro-lycopene) than β-carotene.

According to one embodiment, the Cucumis melo plant comprises only trace amounts of β-carotene.

The melon plant of this aspect of the present invention may comprise a lower level (e.g. 2 fold less) of carotenoid isomerase (CRTISO) mRNA than naturally occurring Cucumis melo plants. Additionally, or alternatively, the melon plant of this aspect of the present invention may comprise a CRTISO with a lower enzymatic activity (e.g. 2 fold less, 5 fold less or 10 fold less) than naturally occurring Cucumis melo plants. According to a particular embodiment, the CRTISO is devoid completely of enzymatic activity.

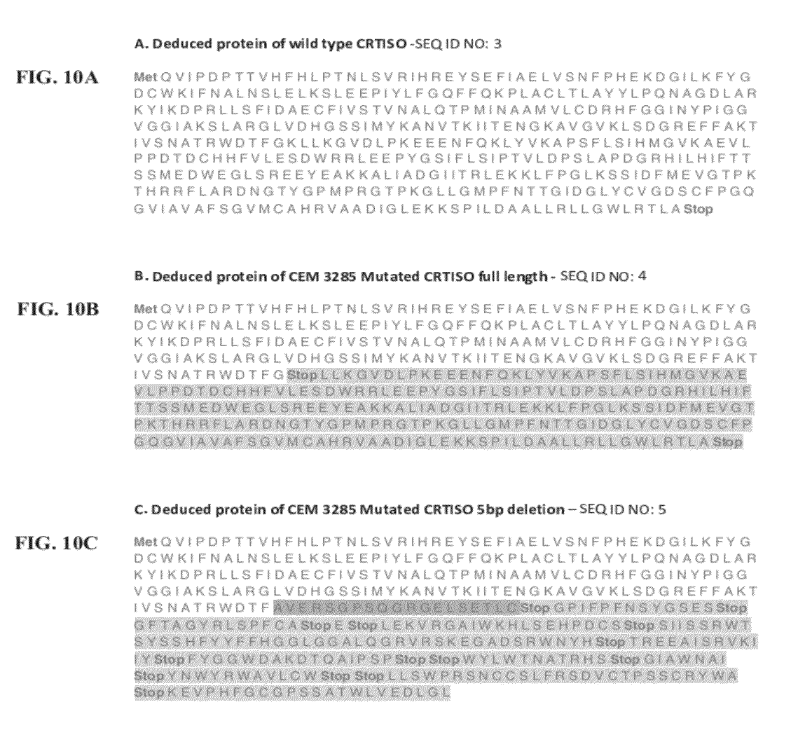

As used herein, the term carotenoid isomerase (CRTISO) refers to the isomerase enzyme (Accession No. IPR014101) that converts tetra-cis-lycopene into all-trans-lycopene (see FIGS. 10A-C).

According to one embodiment, the melon plant of the present invention is devoid of CRTISO catalytic activity.

The present inventors contemplate both chemical mutagenesis and recombinant techniques for the generation of the melon plants of the present invention.

Thus, the melon plants of the present invention may be generated by exposing the melon plant or part thereof to a chemical mutagen. Examples of chemical mutagens include, but are not limited to nitrous acid, alkylating agents such as ethyl methanesulfonate (EMS), methyl methane sulfonate (MMS), diethylsulfate (DES), and base analogs such as 5-bromo-deoxyuridine (5BU). An exemplary method for generating the melon plants of the present invention using chemical mutagenesis includes soaking melon seeds for 12 hours in water followed by additional 12 hours in EMS (e.g. 1%). The treated seeds (M₁) are then planted and self pollinated to prepare M₂ families

Melon plants generated by chemical mutagenesis could comprise at least one allele of CRTISO having a loss of function mutation.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

A “loss-of-function mutation” is a mutation in the sequence of a gene, which causes the function of the gene product, usually a protein, to be either reduced or completely absent. A loss-of-function mutation can, for instance, be caused by the truncation of the gene product because of a frameshift or nonsense mutation. A phenotype associated with an allele with a loss of function mutation is usually recessive but can also be dominant.

It will be appreciated that the present invention also contemplates melon plants wherein both alleles of CRTISO carry a loss-of function mutation. In such instances the CRTISO may be in a homozygous form or in a heterozygous form. According to this embodiment, homozygosity is a condition where both alleles at the CRTISO locus are characterized by the same nucleotide sequence. Heterozygosity refers to different conditions of the gene at the CRTISO locus.

According to one embodiment, the plants of the present invention are of a hybrid variety—i.e. are generated following the crossing (i.e. mating) of two non-isogenic plants. The hybrid may be an F₁ Hybrid or an open-pollinated variety.

An F₁ Hybrid” as used herein, refers to first generation progeny of the cross of two non-isogenic plants.

The development of melon hybrids of the present invention requires the development of homozygous stable parental lines. In breeding programs desirable traits from two or more germplasm sources or gene pools are combined to develop superior breeding varieties. Desirable inbred or parent lines are developed by continuous selfing and selection of the best breeding lines, sometimes utilizing molecular markers to speed up the selection process.

Once the parental lines that give the best hybrid performance have been identified, the hybrid seed can be produced indefinitely, as long as the homogeneity and the homozygosity of the parents are maintained. A single-cross hybrid is produced when two parent lines are crossed to produce the F₁ progeny. Much of the hybrid vigor exhibited by F₁ hybrids is lost in the next generation (F₂). Consequently, seed harvested from hybrid varieties are typically not used for planting stock. According to one embodiment the melon plants of the present invention are stable parent plant lines.

As defined herein, the phrase “stable parental lines” refers to open pollinated, inbred lines, stable for the desired plants over cycles of self-pollination and planting. Typically, 95% or more (e.g. 100%) of the genome is in a homozygous form in the parental lines of the present invention.

According to another aspect, the present invention provides a method for producing first generation (F₁) hybrid melon seeds.

According to one embodiment, the present invention provides a method for producing first generation hybrid seeds comprising crossing a first stable parent melon plant with a second stable parent melon plant and harvesting the resultant hybrid F₁ seeds, wherein the first and the second stabilized parent melon plants have a fruit flesh comprising a greater amount of tetra-cis-lycopene (pro-lycopene) than β-carotene.

According to another embodiment, the present invention also provides a first generation F₁ hybrid melon plants that are produced by growing the hybrid melon seeds produced by the above-described method.

The present invention also relates to seeds harvested from these F₁ hybrid melon plants and plants grown from these seeds.

A common practice in plant breeding is using the method of backcrossing to develop new varieties by single trait conversion.

The phrase “single trait conversion” as used herein refers to the incorporation of new single gene into a parent line wherein essentially all of the desired morphological and physiological characteristics of the parent lines are recovered in addition to the single gene transferred.

The term “backcrossing” as used herein refers to the repeated crossing of a hybrid progeny back to one of the parental melon plants. The parental melon plant which contributes the gene for the desired characteristic is termed the nonrecurrent or donor parent. This terminology refers to the fact that the nonrecurrent parent is used one time in the backcross protocol and therefore does not recur. The parental melon plant to which the gene from the nonrecurrent parent are transferred is known as the recurrent parent as it is used for several rounds in the backcrossing protocol.

In a typical backcross protocol, a plant from the original varieties of interest (recurrent parent) is crossed to a plant selected from second varieties (nonrecurrent parent) that carries the single gene of interest to be transferred. The resulting progeny from this cross are then crossed again to the recurrent parent and the process is repeated until a melon plant is obtained wherein essentially all of the desired morphological and physiological characteristics of the recurrent parent are recovered in the converted plant, in addition to the single transferred gene from the nonrecurrent parent.

Backcrossing methods can be used with the present invention to improve or introduce a characteristic into the parent lines.

It will be appreciated that the present invention also contemplates generating the Cucumis melo fruit by seeding seeds of the melon fruit and/or planting seedlings of the seeds, growing the plants generated from the seeds or seedlings and harvesting the melon fruit of the plants.

As mentioned, the melon plant of the present invention may also be generated using other techniques including but not limited to (a) deletion of the CRTISO gene; (b) transcriptional inactivation of the CRTISO gene (c) antisense RNA mediated inactivation of transcripts of the CRTISO gene; and (d) translational inactivation of transcripts of the CRTISO gene.

Thus, for example, gene knock-in or gene knock-out constructs including sequences homologous with the CRTISO gene can be generated and used to insert an ancillary sequence into the coding sequence of the enzyme encoding gene, to thereby inactivate this gene.

These construct preferably include positive and negative selection markers and may therefore be employed for selecting for homologous recombination events. One ordinarily skilled in the art can readily design a knock-in/knock-out construct including both positive and negative selection genes for efficiently selecting transformed plant cells that underwent a homologous recombination event with the construct. Such cells can then be grown into full plants. Standard methods known in the art can be used for implementing knock-in/knock out procedure. Such methods are set forth in, for example, U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson, Methods in Enzymology, 194:251-270, 1991; Capecchi, Science 244:1288-1292, 1989; Davies et al., Nucleic Acids Research, 20 (11) 2693-2698, 1992; Dickinson et al., Human Molecular Genetics, 2(8):1299-1302, 1993; Duff and Lincoln, “Insertion of a pathogenic mutation into a yeast artificial chromosome containing the human APP gene and expression in ES cells”, Research Advances in Alzheimer's Disease and Related Disorders, 1995; Huxley et al., Genomics, 9:742-750 1991; Jakobovits et al., Nature, 362:255-261 1993; Lamb et al., Nature Genetics, 5: 22-29, 1993; Pearson and Choi, Proc. Natl. Acad. Sci. USA, 1993, 90:10578-82; Rothstein, Methods in Enzymology, 194:281-301, 1991; Schedl et al., Nature, 362: 258-261, 1993; Strauss et al., Science, 259:1904-1907, 1993, WO 94/23049, WO93/14200, WO 94/06908 and WO 94/28123 also provide information.

At the transcription level, expressing antisense or sense oligonucleotides that bind to the genomic DNA by strand displacement or the formation of a triple helix, may prevent transcription. At the transcript level, expression of antisense oligonucleotides that bind target mRNA molecules lead to the enzymatic cleavage of the hybrid by intracellular RNase H or prevention of translation thereof into a protein. In this case, by hybridizing to the targeted mRNA, the oligonucleotides provide a duplex hybrid recognized and destroyed by the RNase H enzyme or which prevents binding to ribosomes. In addition the use of ribozyme sequences linked to antisense oligonucleotides can also facilitate target sequence cleavage by the ribozyme. Alternatively, such hybrid formation may lead to interference with correct RNA splicing into messenger RNA. As a result, in all cases, the number of the target mRNA intact transcripts ready for translation is reduced or eliminated. At the translation level, antisense oligonucleotides or analogs that bind target mRNA molecules prevent, by steric hindrance, binding of essential translation factors (ribosomes), to the target mRNA, a phenomenon known in the art as hybridization arrest, disabling the translation of such mRNAs.

Thus according to a particular embodiment of the present invention, the melon plant is generated by introduction thereto of a nucleic acid construct, the nucleic acid construct comprising a nucleic acid sequence encoding a polynucleotide agent which down-regulates an expression of CRTISO and a cis-acting regulatory element capable of directing an expression of the polynucleotide agent in the plant.

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells. The genetic construct can be an expression vector wherein the nucleic acid sequence is operably linked to one or more regulatory sequences allowing expression in the plant cells.

In a particular embodiment of the present invention the regulatory sequence is a plant-expressible promoter.

As used herein the phrase “plant-expressible” refers to a promoter sequence, including any additional regulatory elements added thereto or contained therein, is at least capable of inducing, conferring, activating or enhancing expression in a melon cell, tissue or organ.

The promoter may be a regulatable promoter, a constitutive promoter or a tissue-associated promoter.

As used herein, the term “regulatable promoter” refers to any promoter whose activity is affected by specific environmental or developmental conditions.

As used herein, the term “constitutive promoter” refers to any promoter that directs RNA production in many or all tissues of a plant transformant at most times.

As used herein, the term “tissue-associated promoter” refers to any promoter which directs RNA synthesis at higher levels in particular types of cells and tissues (e.g., a fruit-associated promoter).

Exemplary promoters that can be used to express an operably linked nucleic acid sequence (i.e. transgene) include the cauliflower mosaic virus promoter, CaMV and the tobacco mosaic virus, TMV, promoter.

Other promoters that can be used in the context of the present invention include those described in U.S. Patent No. 20060168699 and by Hector G. Numez-Palenius et al. [Critical Reviews in Biotechnology, Volume 28, Issue 1 Mar. 2008, pages 13-55], both of which are incorporated herein by reference.

As mentioned, downregulation of CRTISO can be effected on the genomic and/or the transcript level using a variety of molecules which interfere with transcription and/or translation [e.g., RNA silencing agents (e.g., antisense, siRNA, shRNA), Ribozyme and DNAzyme] of CRTISO.

The term “siRNA” refers to small inhibitory RNA duplexes (generally between 18-30 basepairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of the present invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550; SEQ ID NO: 6) and 5′-UUUGUGUAG-3′ (Castanotto, D. et al. (2002) RNA 8:1454, SEQ ID NO: 7). It will be recognized by one of skill in the art that the resulting single chain oligonucleotide forms a stem-loop or hairpin structure comprising a double-stranded region capable of interacting with the RNAi machinery.

According to another embodiment the RNA silencing agent may be a miRNA. miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target) The pre-miRNA is a substrate for a form of dicer that removes the miRNA duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

Unlike, siRNAs, miRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both miRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728). A recent report (Hutvagner et al., 2002, Sciencexpress 297:2056-2060) hypothesizes that gene regulation through the miRNA pathway versus the siRNA pathway is determined solely by the degree of complementarity to the target transcript. It is speculated that siRNAs with only partial identity to the mRNA target will function in translational repression, similar to an miRNA, rather than triggering RNA degradation.

Synthesis of RNA silencing agents suitable for use with the present invention can be effected as follows. First, the CRTISO mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (worldwidewebdotambiondotcom/techlib/tn/91/912.html).

Second, potential target sites are compared to an appropriate genomic database (e.g., human, mouse, rat etc.) using any sequence alignment software, such as the BLAST software available from the NCBI server (wwwdotncbidotnlmdotnihdtgov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

For example, a suitable siRNA that can be used in the context of the present invention is set forth in SEQ ID NO: 8.

It will be appreciated that the RNA silencing agent of the present invention need not be limited to those molecules containing only RNA, but further encompasses chemically-modified nucleotides and non-nucleotides.

In some embodiments, the RNA silencing agent provided herein can be functionally associated with a cell-penetrating peptide.” As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of the present invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of the present invention preferably include, but are not limited to, penetratin, transportan, pIsl, TAT(48-60), pVEC, MTS, and MAP.

Another agent capable of downregulating a CRTISO is a DNAzyme molecule capable of specifically cleaving an mRNA transcript or DNA sequence of the CRTISO. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther worldwidewebdotasgtdotorg). In another application, DNAzymes complementary to bcr-abl oncogenes were successful in inhibiting the oncogenes expression in leukemia cells, and lessening relapse rates in autologous bone marrow transplant in cases of CML and ALL.

Downregulation of a CRTISO can also be effected by using an antisense polynucleotide capable of specifically hybridizing with an mRNA transcript encoding the CRTISO.

Design of antisense molecules which can be used to efficiently downregulate a CRTISO must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

Algorithms for identifying those sequences with the highest predicted binding affinity for their target mRNA based on a thermodynamic cycle that accounts for the energetics of structural alterations in both the target mRNA and the oligonucleotide are available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

A suitable antisense polynucleotide targeted against the mRNA (which is coding for the CRTISO protein) would comprise a sequence as set forth in SEQ ID NO: 13.

Another agent capable of downregulating a CRTISO is a ribozyme molecule capable of specifically cleaving an mRNA transcript encoding a CRTISO. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications.

The constructs of the present invention may also comprise polynucleotide sequences that encode an additional trait (e.g. disease resistance or stress resistance). Such traits may include herbicide resistance, insect resistance, resistance to bacterial, fungal or viral disease, male sterility and improved nutritional value.

Additionally, or alternatively, the constructs of the present invention may comprise polynucleotide sequences that encode selectable markers.

The selectable marker gene can be a gene encoding a neomycin phosphotransferase protein, a phosphinothricin acetyltransferase protein, a glyphosate resistant 5-enol-pyruvylshikimate-3-phosphate synthase (EPSPS) protein, a hygromycin phosphotransferase protein, a dihydropteroate synthase protein, a sulfonylurea insensitive acetolactate synthase protein, an atrazine insensitive Q protein, a nitrilase protein capable of degrading bromoxynil, a dehalogenase protein capable of degrading dalapon, a 2,4-dichlorophenoxyacetate monoxygenase protein, a methotrexate insensitive dihydrofolate reductase protein, and an aminoethylcysteine insensitive octopine synthase protein. The corresponding selective agents used in conjunction with each gene can be: neomycin (for neomycin phosphotransferase protein selection), phosphinotricin (for phosphinothricin acetyltransferase protein selection), glyphosate (for glyphosate resistant 5-enol-pyruvylshikimate-3-phosphate synthase (EPSPS) protein selection), hygromycin (for hygromycin phosphotransferase protein selection), sulfadiazine (for a dihydropteroate synthase protein selection), chlorsulfuron (for a sulfonylurea insensitive acetolactate synthase protein selection), atrazine (for an atrazine insensitive Q protein selection), bromoxinyl (for a nitrilase protein selection), dalapon (for a dehalogenase protein selection), 2,4-dichlorophenoxyacetic acid (for a 2,4-dichlorophenoxyacetate monoxygenase protein selection), methotrexate (for a methotrexate insensitive dihydrofolate reductase protein selection), or aminoethylcysteine (for an aminoethylcysteine insensitive octopine synthase protein selection).

The scoreable marker gene can be a gene encoding a beta-glucuronidase protein, a green fluorescent protein, a yellow fluorescent protein, a beta-galactosidase protein, a luciferase protein derived from a luc gene, a luciferase protein derived from a lux gene, a sialidase protein, streptomycin phosphotransferase protein, a nopaline synthase protein, an octopine synthase protein or a chloramphenicol acetyl transferase protein.

Plant cells may be transformed stably or transiently with the nucleic acid constructs of the present invention. In stable transformation, the nucleic acid molecule of the present invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledenous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by the present invention.

Transient transformation can be effected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of the present invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of the present invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

It is expected that during the life of a patent maturing from this application many relevant techniques for transforming plants will be developed and the scope of the phrase “plant transformation” is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Chemical Mutagenesis of Orange Melon

Materials and Methods

Chemical Mutagenesis:

Seeds of a ‘Charentais’ type melon were exposed to 1% Ethyl methanesulfonate (EMS) for 12 hours. The treated seeds (M₁) were planted and self pollinated to prepare M₂ families

Results

One of the families, CEM 3285, segregated for a recessive altered orange color (FIG. 2).

HPLC analysis of carotenoids in the mutant fruit (CEM 3285) revealed an altered carotenoid pattern compared to wild-type. The major carotenoid in the mutated fruit was tetra-cis-lycopene (pro-lycopene) while the wild-type fruit accumulated β-carotene as the major pigment (FIG. 3). A quarter of the analyzed M2 plants carried mutated fruit indicating the monogenic recessive inheritance of this trait. The HPLC chromatogram of CEM 3285 fruit flesh resembled the carotenoid pattern of tangerine tomato and of pro-lycopene accumulating watermelon.

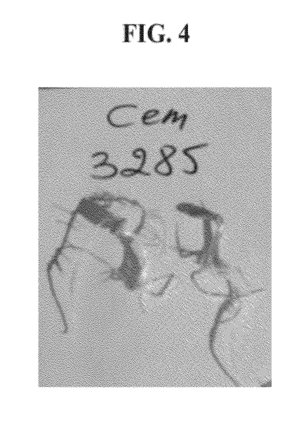

It was observed that CEM 3285 segregated for pale plantlets (FIG. 4) as a recessive monogenic trait. The pale tissue started to accumulate chlorophyll upon exposure to light.

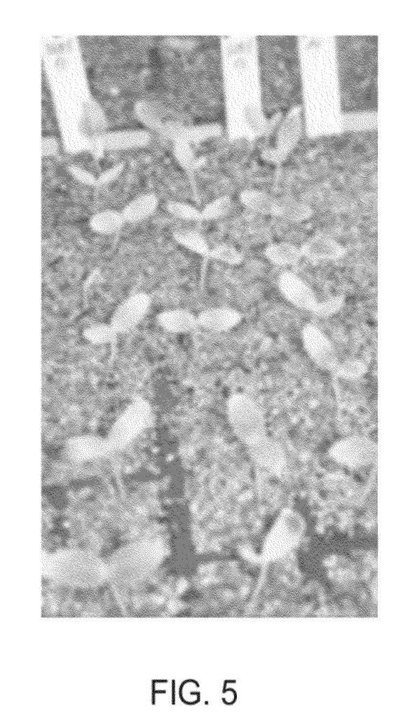

It was further observed that self pollination of plants producing fruits with pro-lycopene yield seeds that emerge as pale plantlets (FIG. 5) and plants that accumulate pro-lycopene as their major fruit carotenoid.

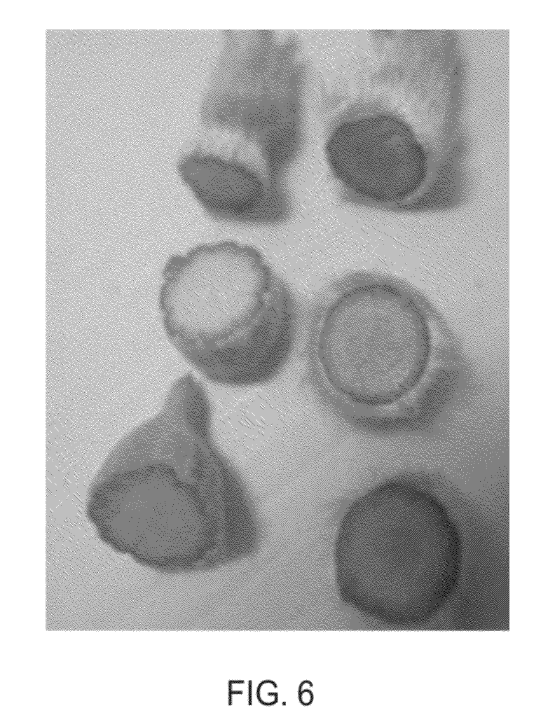

Flowers of lines stabilized for pro-lycopene accumulation (homozygous for the mutation) have petals with altered pale yellow with some orange nuance as compared to the intense yellow petals of the wild-type (FIG. 6).

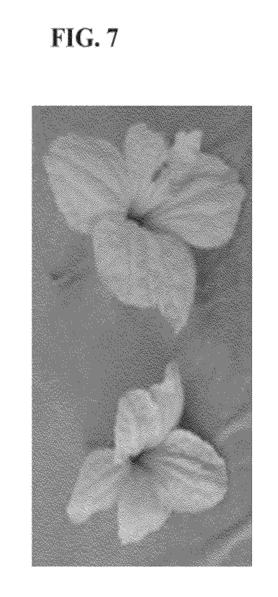

The inner part of the ovaries of the mutated phenotype was orange-yellow as opposed to green of the wild-type (FIG. 7).

### Example 2

### Analysis of DNA Extracted from CEM 3285 Mutant

Materials and Methods

Extraction of Nucleic Acids from CEM 3285 Mutant:

Total genomic DNA was isolated utilizing the CTAB protocol.

Messenger RNA was extracted utilizing the SIGMA's ‘GenElute Mammalian Total RNA Miniprep’ kits.

Conversion of mRNA to cDNA was done with the THERMO's ‘Verso cDNA’ kit.

Selection of primers was assisted with GENERUNNER (v 3.05 Hastings Software) based on published (www.worldwidewebdoticugidotorg) and unpublished sequences of CRTISO in melon and in watermelon.

PCR amplification is conducted utilizing D4309 Sigma REDTaq® DNA Polymerase.

Sequencing of C. melo Carotenoid Isomerase (CRTISO):

Sequencing of CRTISO was performed with the 3130x1 GENETIC ANALYZER of Applied Biosystems utilizing the manufacturer protocols.

qRT-PCR Analysis:

Real-time PCR analyses were performed with the ABI Prism7000 Sequence Detection System (Applied Biosystems, Foster, Calif.). Amplifications were conducted using the ABsoluteTMQPCR SYBR® Green Mixes (ABgene®'s Inc., Epsom, UK). The following primer sequences (0.2 μm final concentration) were used: (1) cyclophiline (a house-keeping gene, accessions no. DV632830) forward primer 5′-GATGGAGCTCTACGCCGATGTC-3′ (SEQ ID NO: 9) and reverse 5′-CCTCCCTGGCACATGAAATTAG-3′ (SEQ ID NO: 10); CRTISO forward primer 5′-AGGGGACTGGTTGATCATGG-3′ (SEQ ID NO: 11) and reverse 5′-GCACAAAATGGTGACAATCTGT-3′ (SEQ ID NO: 12).

Results

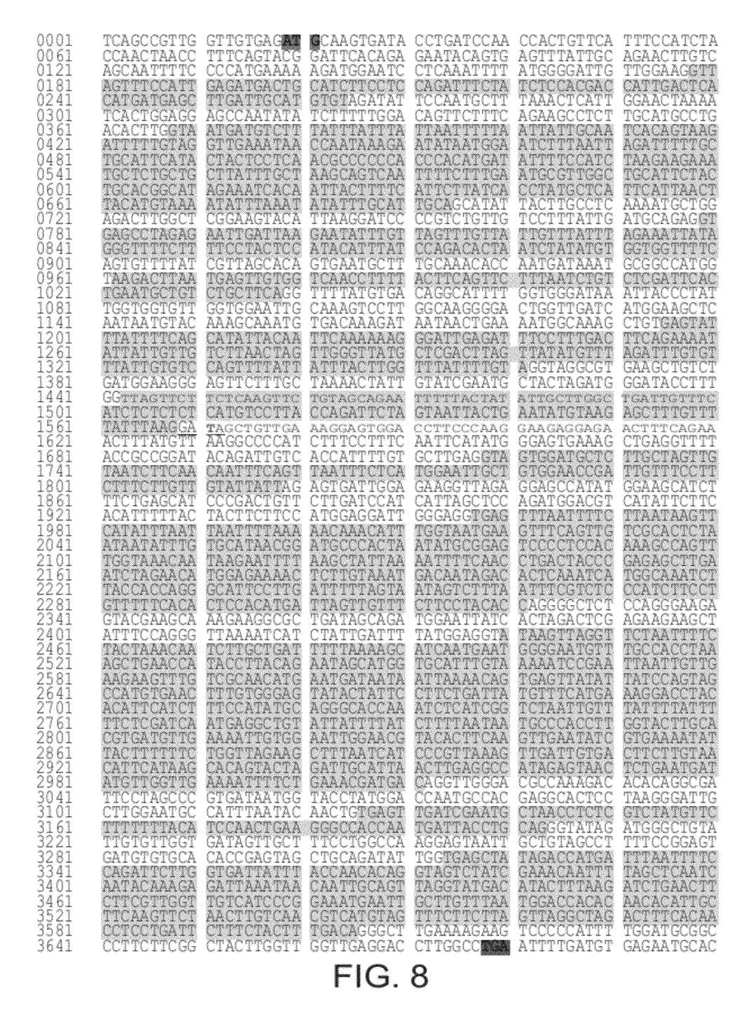

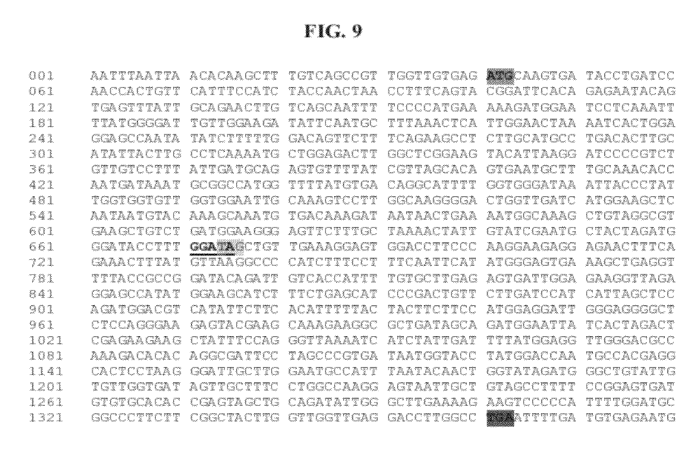

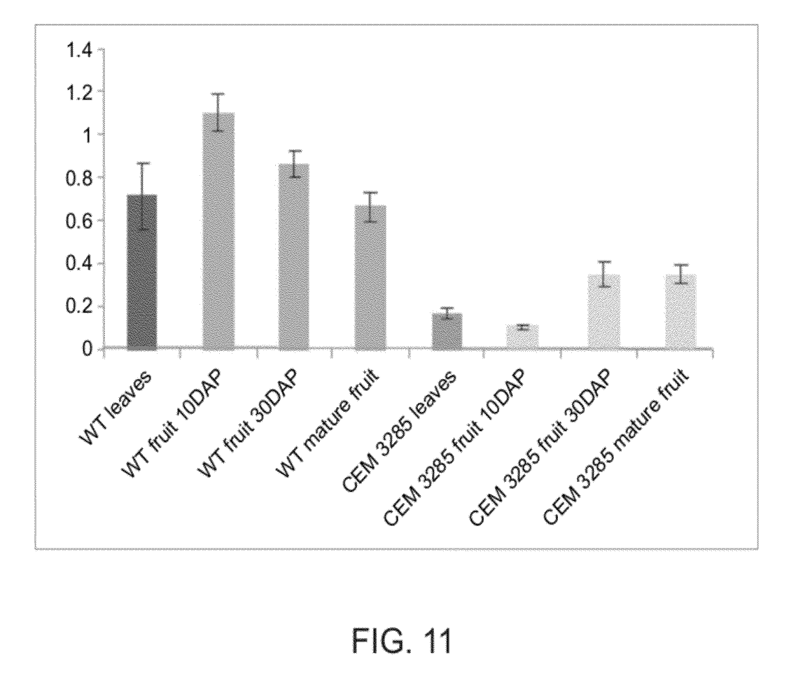

The genomic CRTISO from DNA extracted from CEM 3285 mutants was sequenced and compared to its sequence in wild-type and additional melon lines. All wild-type lines had identical sequences indicating that this gene is highly conserved. An A to T base transversion was observed at position 1554 (position 634 of the cDNA; count starts from the ATG) causing a transition of lysine to a STOP codon (AAG→TAG) following translation. The base transversion occurred in the fourth base of the seventh exon (FIG. 8). Due to the proximity of the induced mutation to the intron-exon junction it also caused mis-splicing of this gene such that two transcripts of CRTISO are expressed, one with the wild-type size and one that carries a deletion of five bases as evidenced by PCR amplification of the mRNA extracted from developing fruits (FIG. 9). The mutated mRNA causes immature STOP codon when full length mRNA is transcribed and both immature STOP codon and alteration of amino acids due to the frameshift mutation caused by the five base pairs deletion (FIGS. 10A-C). qRT-PCR analyses of developing fruits and of leaves indicated significantly lower transcriptional level of CRTISO in the mutated leaves and fruits (FIG. 11).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A Cucumis melo plant, wherein a flesh of a fruit of the plant comprises tetra-cis-lycopene (pro-lycopene).

2. The plant of claim 1, wherein said flesh of said fruit of the plant comprises a greater amount of tetra-cis-lycopene (pro-lycopene) than β-carotene.

3. The plant of claim 1, having a genome, said genome comprises at least one allele of CRTISO having a loss of function mutation.

4. The plant of claim 1, comprising a nucleic acid construct, said nucleic acid construct comprising a nucleic acid sequence encoding a polynucleotide agent which down-regulates an expression of CRTISO and a cis-acting regulatory element capable of directing an expression of said polynucleotide agent in the plant.

5. (canceled)

6. A Cucumis melo plant, having a genome, said genome comprising at least one allele of CRTISO having a loss of function mutation.

7. The plant of claim 6, wherein a flesh of a fruit of said plant comprises pro-lycopene.

8. (canceled)

9. The plant of claim 1, being devoid of carotenoid isomerase catalytic activity.

10. The plant of claim 3, wherein each allele of said CRTISO carries at least one loss of function mutation.

11-12. (canceled)

13. The plant of claim 1, wherein the plant is a stable parental line.

14. The plant of claim 1, wherein the plant is a hybrid generated by crossing two parental lines.

15-22. (canceled)

23. A method for producing a hybrid melon seed comprising crossing a first parent melon plant with a second parent melon plant and harvesting the resultant hybrid F₁ seed, wherein a flesh of a fruit of at least one of the plants comprises tetra-cis-lycopene (pro-lycopene) or a genome of at least one of the plants comprises at least one allele of CRTISO having a loss of function mutation.

24-26. (canceled)

27. A method of generating the plant of claim 1, the method comprising down-regulating an amount and/or activity of carotenoid isomerase (CRTISO) in a Cucumis melo plant, thereby generating the plant.

28. The method of claim 27, wherein said down-regulating is effected by chemical mutagenesis.

29. (canceled)

30. (canceled)

31. A method of generating a Cucumis melo fruit having a flesh which comprises a greater amount of tetra-cis-lycopene (pro-lycopene) than β-carotene and/or having at least one allele of CRTISO having a loss of function mutation, the method comprising:
(a) seeding seeds of the Cucumis melo fruit, and/or planting seedlings of said seeds;
(b) growing plants generated from said seeds or said seedlings; and
(c) harvesting the Cucumis melo fruit of said plants, thereby generating the Cucumis melo fruit.

32. A seed of a Cucumis melo line CEM 3285, a sample of the seed of which has been deposited under NCIMB Accession Number 41710.

33. A plant of Cucumis melo line CEM 3285, a sample of the seed of which has been deposited under NCIMB Accession Number 41710.

34. The plant of claim 6, being devoid of carotenoid isomerase catalytic activity.

35. The plant of claim 6, wherein each allele of said CRTISO carries at least one loss of function mutation.

36. The plant of claim 6, wherein the plant is a stable parental line.

37. The plant of claim 6, wherein the plant is a hybrid generated by crossing two parental lines.
